SEQUENCE LISTING (11 sequences)

SEQ ID NO: 1 - DNA, 18 bp, Artificial sequence
  Primer
gctgctagtgtccgatgt

SEQ ID NO: 2 - DNA, 18 bp, Artificial sequence
  Primer
gatctcctagagtcgtga

SEQ ID NO: 3 - DNA, 20 bp, Artificial sequence
  Partial sensor probe
gcttgaatcgctatccttgm

SEQ ID NO: 4 - DNA, 20 bp, Artificial sequence
  Partial sensor probe
atcctctcctgtaagaacac

SEQ ID NO: 5 - DNA, 24 bp, Artificial sequence
  Detector oligonucleotide
acaggagaggatgcaaggatagcg

SEQ ID NO: 6 - DNA, 20 bp, Artificial sequence
  Partial sensor probe
agcccaccaccagactctts

SEQ ID NO: 7 - DNA, 20 bp, Artificial sequence
  Partial sensor probe
atcgtctttcttagccgccg

SEQ ID NO: 8 - DNA, 24 bp, Artificial sequence
  Detector probe
aagaaagacgatgaagagtctggt

SEQ ID NO: 9 - DNA, 20 bp, Artificial sequence
  Partial sensor probe
gagatgacttcactgtaags

SEQ ID NO: 10 - DNA, 20 bp, Artificial sequence
  Partial sensor probe
ttcggtgatgcaacagcagc

SEQ ID NO: 11 - DNA, 24 bp, Artificial sequence
  Detector probe
tgcatcaccgaagcttacagtgaa

(19) United States
(12) Patent Application Publication
(10) Pub. No.: US 2003/0082584 A1
(43) Pub. Date: May 1, 2003

(54) Enzymatic ligation-based identification of transcript expression

(76) Inventors: Liang Shi, San Diego, CA (US); Bi-Yu Li, San Diego, CA (US); Xun Wang, San Diego, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 10/187,039

(22) Filed: Jun. 28, 2002

Related U.S. Application Data
(60) Provisional application No. 60/302,092, filed on Jun. 29, 2001.
(60) Provisional application No. 60/357,891, filed on Feb. 19, 2002.

(51) Int. Cl. C12Q 1/68; C12P 19/34

(52) U.S. Cl. 435/6; 435/91.2

(57) ABSTRACT

Disclosed herein are novel methods for use in the analysis of nucleic acids. Uses disclosed include polynucleotide expression analysis and detection of single nucleotide polymorphisms. Also provided are diagnostic methods and kits for conducting the disclosed methods.

### CROSS REFERENCE TO RELATED APPLICATIONS

This application claims the benefit of U.S. Provisional Patent Application Serial No. 60/302,092, filed Jun. 29, 2001 and U.S. Provisional Patent Application No. 60/357,891, filed Feb. 19, 2002; each of which is herein incorporated by reference in its entirety for all purposes.

### BACKGROUND

The availability of complete genomic sequences of various organisms promises to significantly advance our understanding of various fundamental aspects of biology. It also promises to provide unparalleled applied benefits such as understanding genetic basis of certain diseases, providing new targets for therapeutic intervention, developing a new generation of diagnostic tests, etc. New and improved tools, however, will be needed to harvest and fully realize the potential of genomics research.

Even though the DNA complement or gene complement is identical in various cells in the body of multi-cellular organisms, there are qualitative and quantitative differences in polynucleotide expression in various cells. A human genome is estimated to contain roughly about 30,000-40,000 genes, however, only a fraction of these genes are expressed in a given cell (International Human Genome Sequence Consortium, Nature, 409:860-921, 2001; Venter et al., Science, 291:1304-1351, 2001). Moreover, there are quantitative differences among the expressed genes in various cell types. Although all cells express certain housekeeping genes, each distinct cell type additionally expresses a unique set of genes. Phenotypic differences between cell types are largely determined by the complement of proteins that are uniquely expressed. It is the expression of this unique set of genes and the encoded proteins, which constitutes functional identity of a cell type, and distinguishes it from other cell types. Moreover, the complement of genes that are expressed, and their level of expression vary considerably depending on the physiological or developmental stage of a given cell type. Certain genes are specifically activated or repressed during differentiation of a cell. The level of expression also changes during development and differentiation. Qualitative and quantitative changes in polynucleotide expression also take place during cell division, e.g. in various phases of cell cycle. Signal transduction by biologically active molecules such as hormones, growth factors and cytokines often involves modulation of polynucleotide expression. Global change in polynucleotide expression also plays a determinative role in the process of aging.

In addition to the endogenous or internal factors as mentioned above, certain external factors or stimuli, such as environmental factors, also bring about changes in polynucleotide expression profile. Infectious organisms such as bacteria, viruses, fungi and parasites interact with the cells and influence the qualitative and quantitative aspects of polynucleotide expression. Thus, precise complement of genes expressed by a given cell type is influenced by a number of endogenous and exogenous factors. The outcome of these changes is critical for normal cell survival, growth, development and response to environment. Therefore, it is important to identify, characterize and measure changes in polynucleotide expression. The knowledge gained from such analysis will not only further our understanding of basic biology, but it will also allow us to exploit it for various purposes such as diagnosis of infectious and non-infectious diseases, screening to identify and develop new drugs, etc.

Besides the conventional, one by one polynucleotide expression analysis methods like Northern analysis, RNase protection assays, and real time PCT (RT-PCR); there are several methods currently available to examine polynucleotide expression in a genome wide scale. These approaches are variously referred to as RNA profiling, differential display, etc. These methods can be broadly divided into three categories: (1) hybridization-based methods such as subtractive hybridization (Koyama et al., Proc. Natl. Acad. Sci. USA 84: 1609-1613, 1987; Zipfel et al., Mol. Cell. Biol. 9: 1041-1048, 1989), microarray (U.S. Pat. No. 6,150,095), etc., (2) cDNA tags: EST, serial analysis of gene expression (SAGE) (see, e.g. U.S. Pat. Nos. 5,695,937 and 5,866,330), and (3) fragment size based, often referred to as gel-based methods where a differential display is generated upon electrophoretic separation of DNA fragments on a gel such as a polyacrylamide gel (described in U.S. Pat. Nos. 5,871,697, 5,459,037, 5,712,126 and PCT publication No. WO 98/51789).

Microarray based gene analysis approach enables working with hundreds of thousands of genes simultaneously rather than one or a few genes at a time. Microarray technology has come at an appropriate time, when entire genomes of humans and other organisms are being worked out. Massive sequence information generated as a result of genome sequencing, particularly human genome sequencing, has created a demand for technologies that provide high-throughput and speed. Microarrays fill this unique niche. Most of the complex physiological processes precede or succeed change in the expression of a large number of genes. Techniques that were available before the advent of microarrays are not suitable to monitor such large-scale changes in gene expression. DNA microarrays offer the opportunity to perform fast, comprehensive, moderately quantitative analyses on hundreds of thousands of genes simultaneously.

A DNA microarray is composed of an ordered set of DNA molecules of known sequences usually arranged in rectangular configuration in a small space such as 1 cm² in a standard microscope slide format. For example, an array of 200×200 would contain 40,000 spots with each spot corresponding to a probe of known sequence. Such a microarray can be potentially used to simultaneously monitor the expression of 40,000 genes in a given cell type under various conditions. The probes usually take the form of cDNA, ESTs or oligonucleotides. Most preferred are ESTs and oligonucleotides in the range of 30-200 bases long as they provide an ideal substrate for hybridization. There are two approaches to building these microarrays, also known as chips, one involving covalent attachment of pre-synthesized probes, the other involving building or synthesizing probes directly on the chip. The sample or test material usually consists of RNA that has been amplified by PCR. PCR serves the dual purposes of amplifying the starting material as well as allowing introduction of fluorescent tags. For a detailed discussion of microarray technology, see e.g., Graves, Trends Biotechnol. 17: 127-134, 1999.

High-density microarrays are built by depositing an extremely minute quantity of DNA solutions at precise location on an array using high precision machines, a number of which are available commercially. An alternative approach pioneered by Packard Instruments, enables deposition of DNA in much the same way that an ink jet printer deposits spots on paper. High-density DNA microarrays are commercially available from a number of sources such as Affymetrix, Incyte, Mergen, Genemed Molecular Biochemicals, Sequenom, Genomic Solutions, Clontech, Research Genetics, Operon, Stratagene and others. Currently, labeling for DNA microarray analysis involves fluorescence, which allows multiple independent signals to be read at the same time. This allows simultaneous hybridization on the same chip with two samples labeled with different fluorescent dyes. The calculation of the ratio of fluorescence at each spot allows determination of the relative change in the expression of each gene under two different conditions. For example, comparison between a normal tissue and a corresponding tumor tissue using the approach helps in identifying genes whose expression is significantly altered. Thus, the method offers a particularly powerful tool when the gene expression profile of the same cell is to be compared under two or more conditions. High-resolution scanners with capability to monitor fluorescence at various wavelengths are commercially available.

As greater information on the genome of species is obtained, new markers in the form of genetic variations or polymorphisms have been identified for various traits. Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequences repeats (SSR) or microsatellites. These repeats can be dinucleotide, trinucleotide, tetranucleotide or pentanucleotide repeats. Polymorphism results from variation in the number of repeated sequences found at a particular locus.

Recently, attention has focused on single nucleotide polymorphisms (SNPs), which are by far the most common source of variation in the genome, as useful genetic markers. SNPs account for approximately 90% of human DNA polymorphism (Collins et al., Genome Res., 8:1229-1231, 1998). SNPs are single base pair positions in genomic DNA at which different sequence alternatives (alleles) exist in a population. The term SNP is not limited to single base substitutions, but also includes single base insertions or deletions. In addition, short insertions or deletions of 10 base pairs or less are also often categorized as SNPs because they are often detected with methodologies used to detect single base polymorphisms.

Nucleotide substitution SNPs are of two types. A transition is the replacement of one purine by another purine or one pyrimidine by another pyrimidine. A transversion is the replacement of a purine for a pyrimidine or vice versa. The typical frequency at which SNPs are observed is about 1 per 1000 base pairs (Li and Sadler, Genetics, 129:513-523, 1991; Wang et al., Science 280:1077-1082, 1998; Harding et al., Am. J. Human Genet., 60:772-789, 1997; Taillon-Miller et al., Genome Res., 8:748-754, 1998). The frequency of SNPs varies with the type and location of the change in question. In base substitutions, two-thirds of the substitutions involve the C←→T (G←→A) type. This variation in frequency is thought to be related to 5-methylcytosine deamination reactions that occur frequently, particularly at CpG dinucleotides. In regard to location, SNPs occur at a much higher frequence in non-coding regions than they do in coding regions.

There are various ways in which SNPs can affect phenotype. Studies have shown that SNPs can cause major changes in structural folds of mRNA that may affect cell regulation (Shen et al., Proc. Natl. Acad. Sci. USA, 96:7871-7876, 1999). When located in a coding region, the presence of a SNP can result in the production of a protein that is non-functional or has decreased function. When present in a non-coding regulatory region, such as a promoter region, the SNP can alter expression of a gene.

Several methods for the detection of SNPs are known in the art. These include multiplexed allele-specific diagnostic assay (MASDA; U.S. Pat. No. 5,834,181), TaqMan assay (U.S. Pat. No. 5,962,233), molecular beacons (U.S. Pat. No. 5,925,517), microtiter array diagonal gel electrophoresis (MADGE, Day and Humphries, Anal. Biochem., 222:389-395, 1994), PCR amplification of specific alleles (PASA, Sommer et al., Mayo Clin. Proc., 64:1361-1372, 1989), allele specific amplification (ASA, Nichols, Genomics, 5:535-540, 1989), allele-specific PCR (Wu et al., Proc. NatL. Acad. Sci. USA, 86:2757-2760, 1989), amplification refractory mutation system (ARMS, Newton et al., Nuc. Acids Res., 17:2503-2516, 1989), bi-PASA (Liu et al., Genome Res., 7:389-398, 1997), ligase chain reaction (LCR, Barany, Proc. Natl. Acad. Sci. USA, 88:189-193, 1991), oligonucleotide ligation assays (OLA, U.S. Pat. No. 5,830,711; Landegren et al., Science, 214:1077-1080, 1988; Samotiaki et al., Genomics, 20:238-242, 1994; Day et al., Genomics, 29:152-162, 1995; Grossman et al., Nuc. Acids Res., 22:4527-4534, 1994), dye-labeled oligonucleotide ligation (U.S. Pat. No. 5,945,283; Chen et al., Genome Res., 8:549-556, 1998), restriction fragment length polymorphism (RFLP, U.S. Pat. Nos. 5,324,631 and 5,645,995), MALDI-TOF (Bray et al., Hum. Mutat., 17:296-304, 2001), Invader Assay (Hsu et al., Clin. Chem, 47:1373-1377, 2001) and minisequencing either alone (U.S. Pat. Nos. 5,846,710 and 5,888,819; Syvanen et al., Am. J. Hum. Genet., 52:46-59, 1993) or in combination with microarrays (Shumaker et al., Human Mut., 7:346-354, 1996) or fluorescence resonance energy transfer (U.S. Pat. No. 5,945,283; Chen et al., Proc. Natl. Acad. Sci. USA, 94:10756-10761, 1997).

One methodology which has shown promise is microsphere-based liquid arrays. Microsphere-based liquid arrays use different coded microspheres to anchor molecular probes and capture target molecules quantitatively. The captured target molecules are then identified, sorted, and quantified by flow cytometry or other readout methods (Bruchez et al., Science, 281:2013-2016, 1998; Steemers, et al., Nature Biotechnology, 18:91-94; Kettman, et al., Cytometry, 33:234-243, 1998). The Luminex liquid array system, as an example, uses 100 different types of inexpensive, color-coded microspheres to capture and sort target molecules, with a readout speed of 20,000 microspheres/second (Kettman, et al., Cytometry, 33:234-243, 1998). Thus, it provides an inexpensive, multiplexing, high-throughput readout platform (Smith et al., Clinical Chemistry, 44:2054-2056, 1998), and has been widely used in detection of protein ligands (Willman, et al., Am. J. Clin. Pathol., 115:764-769, 2001) and DNA polymorphism in a high-throughput fashion (Ye, et al., Human Mutation, 17:305-316 2001). Recently this system was applied to monitor multiple gene expression simultaneously (Yang, et al., Genome Research, 11:1888-1898, 2001). By sequence specific hybridization, fluorescently labeled nucleic acid target molecules in a biological sample were captured by the DNA oligonucleotide probes anchored at microspheres, and sorted and quantified. However, the assay only detects medium and highly abundant transcripts and it depends on the lengthy and expensive in vitro transcription to amplify the target signals. The low sensitivity and high cost of the target labeling reaction hamper the broad application of the assay, especially for high-throughput screening.

As the amount of genetic information available continues to grow, the need for rapid, cost effective methods of mass gene expression and SNP analysis also grows. The wide scale application of many available methods is limited by high costs associated with consumables used, instrumentation required, the amount of labor involved, or some combination of these three factors. What is need therefore, are methods for nucleic acid analysis that allow for mass screening in a cost effective manner. The present inventors have developed a novel methodology called ELITE (Enzymatic Ligation-based Identification of Transcript Expression), to translate the presence and concentration information of RNA transcripts into quantitative oligonucleotide ligation as well as detect the presence of single nucleotide polymorphisms. The method combines the benefits of the inexpensive T4 DNA ligase in its property to mediate RNA-templated DNA oligonucleotide ligation, the PCR in signal amplification and the liquid array in its multiplexing flexibility and high-throughput readout to achieve sensitive and multiplex detection of polynucleotide expression.

### SUMMARY

Among the several aspects of the invention is provided, a method for determining polynucleotide expression comprising providing a population of test polynucleotides containing one or more target polynucleotides of interest, where at least a portion of the nucleotide sequence of the target polynucleotides is known. For each target polynucleotide, a pair of specific sensor probes are provided, where the first probe of the pair has a 3′ portion that is complementary to the target polynucleotide and a 5′ portion comprising a primer binding site common to all first sensor probes; and the second probe of the pair has a 5′ portion complementary to the target polypeptide and a 3′ portion comprising a primer binding site common to all second sensor probes. In one embodiment, the 5′ end of the second probe is phosphorylated. The sensor probes are such that when hybridized to the target polynucleotide, the 3′ end of the first probe is immediately adjacent to the 5′ end of the second probe, with no gap in between the two probes. If necessary, the target polynucleotides are denatured, then combined with sets of sensor probes under moderately stringent, stringent, or highly stringent conditions and the sensor probes are allowed to hybridize to the target polynucleotides.

After hybridization, a ligase reaction is conducted so the hybridized pairs of sensor probes are ligated to form a ligated sensor probe comprising both members of the sensor probe pair. In one embodiment, a T4 ligase is used. In another embodiment, a single mismatch or more at the 3′ end of the first probe or the 5′ end of the second probe results in a failure of the ligation reaction. The ligated sensor probes are amplified using any suitable method so that the resulting amplified ligated sensor probes contain a detectable label. In one embodiment, amplification is accomplished using the polymerase chain reaction and the common primer binding sites on the sensor probes. In another embodiment, the detectable label is incorporated into the amplified sensor probes using labeled dNTPs, while in still another embodiment, labeled primers are used.

For each different type of pairs of sensor probes, at least one class of detector oligonucleotides is provided. The detector oligonucleotide comprises a detectable label that is unique for that class of detector oligonucleotide and that differs, i.e. is capable of being differentiated, from the detectable label of the sensor probes. At least a portion of the detector oligonucleotide is capable of hybridizing to the ligated and amplified sensor probes. In one embodiment, the detector oligonucleotides further comprise a linker which does not hybridize to the ligated sensor probes, but links the detector oligonucleotide to a label, such as a microparticle, for example a fluorescent microbead or microsphere. In one embodiment, the fluorescent microbead or microsphere is a Luminex microsphere.

The labeled amplified ligated sensor probes are combined with detector oligonucleotides and allowed to hybridize under moderately stringent, stringent, or highly stringent conditions. If necessary, the amplified sensor probes are denatured prior to hybridization. In one embodiment, the detector oligonucleotides hybridize to the ligated sensor probes at a location that spans the ligation site of the sensor probe pair. The hybridization of the detector oligonucleotides to the corresponding ligated amplified sensor probes is determined using any suitable means. In one embodiment, hybridization of the detector oligonucleotide to the amplified ligated sensor probes is determined by detecting the presence of the sensor probe label in association with the detector oligonucleotide label. In one embodiment, the detection is by use of a flow cytometer. If desired, the determination of hybridization can be quantitative.

Another aspect provides detection of a single nucleotide polymorphism of interest comprising providing a test population of polynucleotides which contains at least one target polynucleotide known, or suspected, to contain a single nucleotide polymorphism (SNP). For each target polynucleotide, at least one pair of specific sensor probes is provided where the first sensor probe of the pair has a 3′ end portion that is complementary to the target polynucleotide and a 5′ end portion that contains a common primer binding site; and the second sensor probe of the pair has a 5′ end portion that is complementary to target polynucleotide and a 3′ end portion that contains a common primer binding site. The sensor probes in a pair are such that the 3′ end of the first probe hybridizes on the target polynucleotide immediately adjacent to the 5′ end of the second probe and that either the 3′ terminal nucleotide on the first probe or the 5′ terminal nucleotide on the second probe is complementary to an allele of the SNP of interest. In one embodiment, a second pair of sensor probes is provided. This second pair of sensor probes has the same characteristics as the first pair and hybridizes to the target polynucleotide at the same location, but either the 3′ terminal nucleotide of the first probe in this second pair or the 5′ terminal nucleotide of the second probe of this second pair is complementary to an allele of the SNP of interest that is different from the complementary allele for the first pair of sensor probes

The target polynucleotides are combined with the sensor probe pairs under moderately stringent, stringent, or highly stringent conditions and the sensor probes are allowed to hybridize to the target polynucleotides. If necessary, the target polynucleotides are denatured prior to hybridization. The hybridized sensor probe pairs are ligated to form a ligated sensor probe under conditions such that if there is a mismatch at the site of the SNP of interest, ligation does not occur. That is, if the SNP allele complementary to either the 3′ terminal nucleotide of the first sensor probe, or the 5′ terminal of the second sensor probe is not present, ligation does not occur. In one embodiment, a T4 ligase is used. The successfully ligated sensor probes are amplified by any suitable means to produce amplified ligated sensor probes such that the amplified sensor probes comprise a detectable label. In one embodiment, amplification is achieve by the polymerase chain reaction and the common primer binding sites on the sensor probes. In another embodiment, the detectable label is incorporated into the amplified ligated sensor probes using labeled dNTPs, while in still another embodiment, labeled primers are used.

For each type of sensor probe pair, at least one class of detector oligonucleotide is provided where each class of detector oligonucleotide has a unique detectable label that is different from the detectable label of the amplified sensor probes. At least a portion of the detector oligonucleotide is capable of hybridizing to the ligated and amplified sensor probes. In one embodiment, the detector oligonucleotides further comprise a linker which does not hybridize to the ligated sensor probes, but links the detector oligonucleotide to a label, such as a microparticle, for example a fluorescent microbead or microsphere. In one embodiment, the fluorescent microbead or microsphere is a Luminex microsphere. The detector oligonucleotides and the amplified, ligated sensor probes are combined and allowed to hybridize under moderately stringent, stringent or highly stringent conditions. If necessary, the amplified ligated sensor probes are denatured prior to hybridization. In one embodiment, the detector oligonucleotide hybridize to the ligated sensor probes at a location that spans the ligation site of the sensor probe pair, that is, spans the location of the SNP of interest.

The hybridization of the detector oligonucleotides to the corresponding ligated amplified sensor probes is determined using any suitable means. In one embodiment, hybridization of the detector oligonucleotide to the amplified ligated sensor probes is determined by detecting the presence of the sensor probe label in association with the detector oligonucleotide label. In one embodiment, the detection is by use of a flow cytometer. If desired, the determination of hybridization can be quantitative.

Another aspect provides a method for determining the physiological or developmental state of a cell or tissue. This aspect comprises obtaining a population of polynucleotides from a test cell or tissue comprising at least one target polynucleotide. In one embodiment, the target polynucleotide is isolated from the test cell or tissue using standard methods. The expression of the target polynucleotide or polynucleotides is determined using the methods described herein and the results compared to those obtained using the same method obtained from a reference cell or tissue of a known physiological or developmental state.

Still another aspect provides a method for diagnosing a disease condition, disorder, or predispostion of interest. This aspect comprises obtaining a population of polynucleotides from a cell or tissue of a test subject comprising at least one target polynucleotide. In one embodiment, the target polynucleotide is isolated from the cell or tissue using standard methods. The expression of the target polynucleotide or polynucleotides is determined using the methods described herein and the results compared to those obtained using the same method obtained from a cell or tissue of a reference subject known to have the disease, condition, disorder, or predisposition of interest.

Yet another aspect provides a method for diagnosing a disease, condition, disorder or predisposition associated with a single nucleotide polymorphism (SNP). This aspect comprises obtaining a population of polynucleotides from a cell or tissue of a test subject and determining the presence, absence, or frequency of at least one allele of at least one SNP of interest using any of the methods described herein. The presence, absence or frequency of the SNP allele of interest in the test subject is compared to the presence, absence or frequency of the allele in a population of polynucleotides from a cell or tissue obtained from a reference subject known to have said disease, condition, disorder, or predisposition.

A further aspect provides kits for practicing the methods disclosed herein. The kits comprise, at a minimum, at least one pair of sensor probes for at least one target polynucleotide of interest, at least one detector oligonucleotide for each pair of sensor probes provided, and instructions for carrying out any of the methods described herein. In one embodiment, the detector oligonucleotides further comprise a microbead or microsphere. The kits may also comprise a ligase, primers, dNTPs, a polymerase and/or buffer solutions.

### BRIEF DESCRIPTION OF THE FIGURES

These and other features, aspects, and advantages of the present invention will become better understood with regard to the following description, appended claims and accompanying figures where:

FIG. 1 shows a depiction of one embodiment of the present invention.

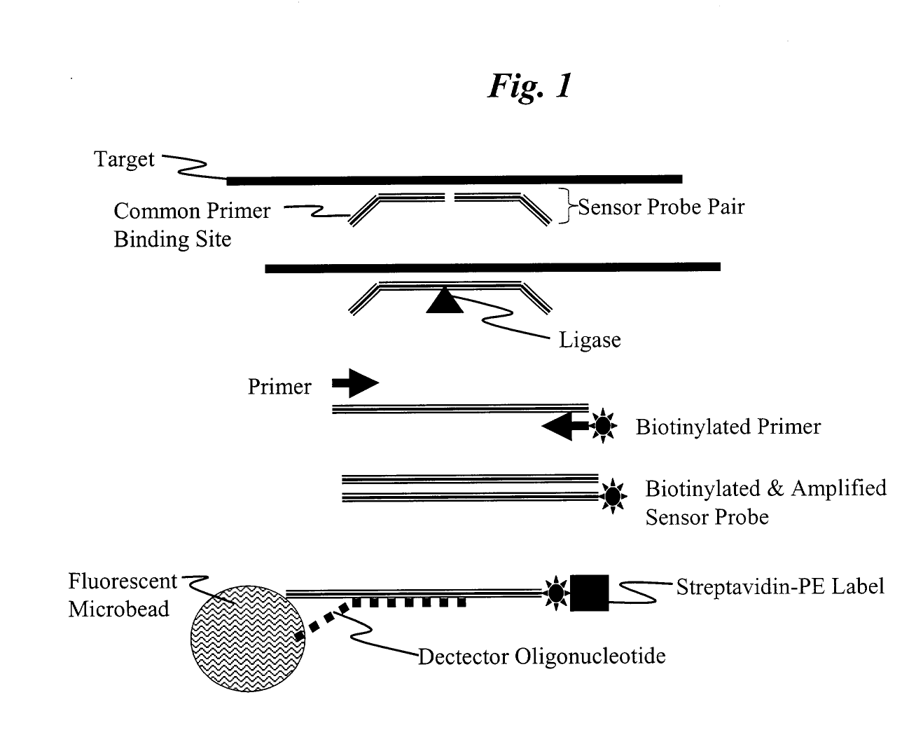

FIG. 2 shows the sensitivity of the methods disclosed for polynucleotide expression analysis.

FIG. 3 shows the dynamic range of the methods disclosed for polynucleotide expression analysis.

FIG. 4 shows the reproducibility of the methods disclosed for polynucleotide expression analysis.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, public databases, public database entries and other references cited in this application are herein incorporated by reference in their entirety as if each individual publication, patent, patent application, public database, public database entry or other reference were specifically and individually indicated to be incorporated by reference.

Aspects of the present invention provide novel methods for use in the analysis of nucleic acids. These methods are particularly useful for expression analysis, such as gene expression analysis. Additional aspects provide methods for the detection of single nucleotide polymorphisms (SNPs). SNPs have a wide variety of uses including diagnosis of genetic diseases and predispositions in plants and animals, including humans, as well as uses to identify valuable phenotypes and in marker assisted selection. In addition to providing multiplex capabilities, the methods provided have the advantages of adaptability, easy of use, and cost effectiveness.

As used herein, “SNP” means single nucleotide polymorphism.

As used herein, “MFI” means mean fluorescence intensity.

As used herein “polynucleotide” and “oligonucleotide” are used interchangeably and refer to a polymeric (2 or more monomers) form of nucleotides of any length, either ribonucleotides or deoxyribonucleotides. Whether or not specifically stated, polynucleotides and oligonucleotides are considered to have a 5′ end and a 3′ end. Although nucleotides are usually joined by phosphodiester linkages, the terms also include peptide nucleic acids such as polymeric nucleotides containing neutral amide backbone linkages composed of aminoethyl glycine units (Nielsen et al., Science, 254:1497, 1991). The terms refer only to the primary structure of the molecule. Thus, the terms include double- and single-stranded DNA and RNA as well DNA/RNA hybrids that may be single-stranded, but are more typically double-stranded. In addition, the terms also refer to triple-stranded regions comprising RNA or DNA or both RNA and DNA. The strands in such regions may be from the same molecule or from different molecules. The regions may include all or one or more of the molecules, but more typically involve only a region of some of the molecules. The terms also include known types of modifications, for example, labels, methylation, “caps”, substitution of one or more of the naturally occurring nucleotides with an analog, internucleotide modifications such as, for example, those with uncharged linkages (e.g. methyl phosphonates, phophotriesters, phosphoamidates, carbamates etc.), those containing pendant moieties, such as, for example, proteins (including for e.g., nucleases, toxins, antibodies, signal peptides, poly-L-lysine, etc.), those with intercalators (e.g., acridine, psoralen, etc,), those containg alkylators, those with modified linkages (e.g. alpha anomeric nucleic acids, etc.), as well as unmodified forms of the polynucleotide. Polynucleotides include both sense and antisense, or coding and template strands. The terms include naturally occurring and chemically synthesized molecules.

The term “detectable label” refers to a label which, when attached, provides a means of detection. There are a wide variety of labels available for this purpose including, without limitation, radioactive labels such as radionuclides, fluorophores or fluorochromes, peptides, enzymes, antigens, antibodies, vitamins or steroids. For example, radioactive nuclides such as ³²P or 35S, or fluorescent dyes are conventionally used to label PCR primers. Chemiluminescent dyes can also be used for the purpose. The label can be attached directly to the molecule of interest or be attached through a linker. More specific examples of suitable labels include xanthine dyes, rhodamine dyes, naphthylamines, benzoxadiazoles, stilbenes, pyrenes, acridines, Cyanine 3, Cyanine 5, phycoerythrin conjugated streptavidin, Alexa 532, fluorescein, tetramethyl rhodamine, fluorescent nucleotides, digoxigenin, and biotin-deoxyuracil triphosphate. Likewise, in some embodiments, the nucleic acid can be labeled using intercalating dyes such as, for example, YOYO, TOTO, Picogreen, ethidium bromide, and the like.

As used herein “sequence” means the linear order in which monomers occur in a polymer, for example, the order of amino acids in a polypeptide or the order of nucleotides in a polynucleotide.

As used herein the terms “microsphere” and “microbead” are used interchangeably.

As used herein, the term “subject” refers to any plant or animal.

As used herein, the term “animal” includes human beings.

As used herein, “primer” or “oligonucleotide primer” or “PCR primer” means an oligonucleotide, either naturally occurring, as in a purified restriction enzyme digest, or produced synthetically, that under the proper conditions, is capable of hybridizing to a template DNA or RNA molecule to initiate primer extension by polymerization, such as by a DNA-dependent DNA polymerase, an RNA-dependent RNA polymerase, or an RNA-dependent DNA polymerase, to produce a DNA or RNA molecule that is complementary to the template molecule. Primers are often between about 5 to about 50, typically between about 10 to about 30 and more typically between about 18 to about 25 nucleotides in length, and do not contain palindromic sequences or sequences resulting in the formation of primer dimers. Often primers are single stranded, however, double stranded primers may be used provided the primer is treated to separate the strands prior to being used for primer extension.

In some aspects of the invention, a target polynucleotide is provided. The target polynucleotide can be DNA or RNA. Any of the various types of DNA and RNA can be used, for example, mRNA, cRNA, viral RNA, synthetic RNA, cDNA, genomic DNA, viral DNA, plasmid DNA, synthetic DNA, amplified DNA or any combination thereof. The target polynucleotides can be obtained from any source containing nucleic acids. Sources typically include cells and tissues from prokaryotes and eukaryotes such as bacteria, yeast, fungi, plants and animals. The target polynucleotides can also be obtained from viruses. By tissue is meant a plurality of cells that in their native state are organized to perform one or more specific functions. Non-limiting examples of tissues include muscle tissue, cardiac tissue, nervous tissue, leaf tissue, stem tissue, root tissue, etc. Cells from which target polynucleotides are obtained can be haploid, diploid, or polyploid.

As used herein, “hybridization” refers to a process in which a strand of nucleic acid joins with a complementary strand through base pairing. Techniques and conditions for hybridization are well known to practitioners in the field. It is also well known in the field that when double stranded nucleic acid molecules are used in hybridization, the double stranded molecules are made typically single stranded, generally by heat or chemical denaturation, prior to hybridiziation. As used herein, hybridization includes such denaturation, if required, whether or not such denaturation is specifically mentioned.

One aspect provides a method for determining expression of a polynucleotide, for example, gene expression. The determination can be, if desired, quantitative. In this method, a population of test polynucleotides, for example DNA or RNA, is provided. The population of polynucleotides can be obtained from any source. For example, the polynucleotides can be obtained from plant or animal cells or tissues. Typically, the polynucelotides are isolated or purified. Methods for the isolation or purification of polynucleotides are well known in the art and can be found in standard texts such as Sambrook et al., Molecular Cloning, 2^{nd} ed., Cold Spring Harbor Laboratory Press, 1989.

The population of polynucleotides contains one or more target polynucleotides of interest where the target polynucleotides comprise a known nucleic acid sequence. In one embodiment, the population of polynucleotides contains at least 20 different target polynucleotides, in another embodiment at least 50 different target polynucleotides, and in still another embodiment at least 100 different polynucleotides. As will be apparent to those skilled in the art, the sequence of all polynucleotides in the population need not be known. Likewise the complete sequence of the target polynucleotides need not be known, but rather only a portion of the sequence of the target polynucleotide need be known.

For each target polynucleotide, at least one pair of specific sensor probes is provided. The specific sensor probes are chosen so that under the conditions used, the sensor probes will preferably hybridize to only one target molecule in the population. It will be apparent that, if desired, more that one pair of sensor probes can be used. That is, for a particular target polynucleotide, different sensor probe pairs that hybridize at different locations on the target polynucleotide or hybridize under different conditions can be used. Each pair of sensor probes are selected so that a 3′ portion of one member of the pair is complementary to the target polynucleotide while the other member of the pair has a 5′ portion that is complementary to the target polynucleotide. The sensor probes are also chosen so that when hybridized to the target polynucleotide, the 3′ end of one member of the pair is immediately adjacent to the 5′ end of the other member of the pair. By immediately adjacent is meant that there is no gap between the 3′ end of one member of the pair and the 5′ end of the other member of the pair. In one embodiment, each sensor probe contains three portions: a first portion that is complementary to the target polynucleotide and to a detector oligonucleotide; a second portion that is complementary to the target polynucleotide, but not the detector oligonucleotide; and a third portion comprising a common primer binding site that is not complementary to either the target polynucleotide or the detector oligonucleotide. In this embodiment, the sensor probes are selected so that when hybridized to the target polynucleotide, the first portion of each member of a pair will be immediately adjacent to each other. Common primer binding sites are portions of the sensor probes which have little or no homology to known nucleotide sequences from the organism from which the target polynucleotides are obtained. The common primer regions may or may not be homologous to the detector oligonucleotides. In one embodiment, the common primer binding sites are common to all sets of sensor probes. The use of common primer binding sites allows the use of the same primers for amplification of all sensor probes.

In one embodiment, the portion of the sensor probe that is complementary to the target polynucleotide is about 15 to about 30 nucleotides in length, in another embodiment about 18 to about 24 nucleotides in length, while in still another embodiment about 20 nucleotides in length. In one embodiment, the common primer binding site of the sensor probes is about 12 to about 24 nucleotides long, in another embodiment about 15 to about 20 nucleotides long and in still another embodiment about 18 nucleotides long.

The population of polynucleotides containing the target polynucleotides of interest are then combined with the pairs of specific sensor probes under conditions that allow for hybridization of the sensor probes to the target polynucleotides. If necessary, prior to hybridization the target polynucleotides are denatured using any means known in the art, for example heat or chemical denaturation. The hybridization conditions can be moderately stringent, stringent or highly stringent. In one embodiment, hybridization is carried out under stringent conditions.

As is well known in the art, stringency is related to the Tₘ of the hybrid formed. The Tₘ (melting temperature) of a nucleic acid hybrid is the temperature at which 50% of the bases are base-paired. For example, if one the partners in a hybrid is a short oligonucleotide of approximately 20 bases, 50% of the duplexes are typically strand separated at the Tₘ. In this case, the Tₘ reflects a time-independent equilibrium that depends on the concentration of oligonucleotide. In contrast, if both strands are longer, the Tₘ corresponds to a situation in which the strands are held together in structure possibly containing alternating duplex and denatured regions. In this case, the Tₘ reflects an intramolecular equilibrium that is independent of time and polynucleotide concentration.

As is also well known in the art, Tₘ is dependent on the composition of the polynucleotide (e.g. length, type of duplex, base composition, and extent of precise base pairing) and the composition of the solvent (e.g. salt concentration and the presence of denaturants such formamide). One equation for the calculation of Tₘ can be found in Sambrook et al. (Molecular Cloning, 2^{nd} ed., Cold Spring Harbor Press, 1989) and is:

Tₘ=81.5° C.−16.6(log₁₀[Na⁺])+0.41(% G+C)−0.63(% formamide)−600/L)

Where L is the length of the hybrid in base pairs, the concentration of Na⁺ is in the range of 0.01M to 0.4M and the G+C content is in the range of 30% to 75%. Equations for hybrids involving RNA can be found in the same reference. Alternative equations can be found in Davis et al., Basic Methods in Molecular Biology, 2^{nd} ed., Appleton and Lange, 1994, Sec 6-8.

Methods for hybridization and washing are well known in the art and can be found in standard references in molecular biology such as those cited herein. In general, hybridizations are usually carried out in solutions of high ionic strength (6× SSC or 6× SSPE) at a temperature 20-25° C. below the Tₘ. Specific examples of stringent hybridization conditions include 5× SSPE, 50% formamide at 42° C. or 5× SSPE at 68° C. Stringent wash conditions are often determined empirically in preliminary experiments, but usually involve a combination of salt and temperature that is approximately 12-25° C. below the Tₘ. One example of highly stringent wash conditions is 1× SSC at 60° C. An example of very highly stringency wash conditions is 0.1× SSPE, 0.1% SDS at 42° C. (Meinkoth and Wahl, Anal. Biochem., 138:267-284, 1984). An example of extremely stringent wash conditions is 0.1× SSPE, 0.1% SDS at 50-65° C. As is well recognized in the art, various combinations of factors can result in conditions of substantially equivalent stringency. Such equivalent conditions are within the scope of the present inventive discovery.

Following hybridization of the sensor probes to the target polynucleotides, pairs of sensor probes that have hybridized to their target polynucleotide immediately adjacent to each other are ligated together to form a ligated sensor probe. Conditions for ligation used as such that the presence of a single mismatch between the adjacent 3′ and 5′ ends of a pair of sensor probes will prevent ligation from occurring. Any suitable ligase known in the art can be used. In one embodiment, a T4 ligase is used. It has long been recognized in the art that T4 DNA ligase can mediate ligation of DNA oligonucleotides hybridized to RNA (Kleppe, et al., Proc. Natl. Acad. Sci. USA, 67:68-73, 1970) and that this ligation permits discrimination of single nucleotide mismatches between the target and the probe (Nilsson, et al., Nature Biotech. 18:791-793, 2000).

The ligated sensor probes are amplified to produce amplified sensor probes. Any known method of amplification can be used including, but not limited to, the polymease chain reaction (PCR) (U.S. Pat. Nos. 4,965,188; 4,800,159; 4,683,202; 4,683,195), ligase chain reaction (Wu and Wallace, Genomics, 4:560-569, 1989; Landegren et al., Science, 241:1077-1080, 1988), transcription amplification (Kwoh et al. Proc. Natl. Acad. Sci. USA, 86:1173-1177, 1989), self-sustained sequenced replication (Guatelli et al., Proc. Natl. Acad. Sci. USA, 87:1874-1878, 1990) and nucleic acid based sequence amplification (NASBA). In one embodiment, amplification is accomplished by PCR.

When primer based amplification such as PCR is used, the common primer binding sites located on the sensor probes can be utilized. Because the primer sites are common across pairs of sensor probes directed to different target molecules, a single primer or set of primers can be used. This limits the bias in amplification that can result due to differences in the efficiency of binding of different primers under the same binding conditions. The lessening or elimination of this bias allows for the amplification to be quantitative. The presence of two primer binding sites on ligated sensor probes allows for preferential amplification of ligated sensor probes. Thus, ligated sensor probes are amplified exponentially, while non-ligated sensor probes are not amplified or are amplified only linearly.

In one embodiment, the amplified sensor probes comprise a detectable label or marker. In one embodiment, the detectable label or marker is incorporated into the amplified sensor probes during amplification. In this embodiment, the label can be incorporated by using primers, one or both of which contain a label; labeled nucleoside triphosphates (NTPs); or a combination of labeled primers and NTPs. Those skilled in the art know which label should be used in conjunction with the particular experimental conditions employed. In certain embodiments, the labels are biotin-deoxyuracil triphosphate and phycoerythrin conjugated streptavidin.

The present invention also provides for each different pair of sensor probes at least one class of detector oligonucleotide that is capable of identifying sensor probes for a particular target polynucletotide. In one embodiment, the ability of the detector oligonucleotide to identify a particular group of sensor probes is due to the presence of a detectable label or maker associated with a particular class of detector oligonucleotide. The detectable label may be coupled directly to the detector oligonucleotide or by means of a linker molecule. In this embodiment, the detectable label is different and so unique for each class of detector oligonucleotide used. Thus, for example, in an experiment where 20 target polynucleotides are detected, at least 20 classes of detector oligonucleotides, each class comprising a different label, are used. The detectable labels or makers used with detector oligonucleotides should be different from the detectable label or marker used with the amplified sensor probes. By different is meant that the labels are capable of being differentiated from each other, for example on the basis of their emission spectra. In one embodiment, detector oligonucleotides are about 18 to about 30 nucleotides in length. In another embodiment, detector oligonucleotides are about 24 nucleotides long.

In one embodiment, the detector oligonucleotide further comprises a microbead or microsphere. In one embodiment, the microbead is attached to the detector oligonucleotide by means of a linker, for example a 5′ amino-unilinker (Oligo Etc., Wilsonville, Oreg.). When microbeads or microspheres are used, the identity of the detector oligonucleotide can be accomplished using microbeads or microspheres of different sizes, shapes and/or colors (labels). The microbeads can range in size from about 0.1 micrometers to about 1000 micrometers, generally about 1 to about 100 micrometers, typically about 2 to about 50 micrometers, more typically about 3 to about 25 micrometers, usually about 6 to about 12 micrometers. The microbeads can be made of any suitable material including, but not limited to, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, polysulfone, or combinations thereof. Other polymer materials such as carbohydrate, e.g., carboxymethyl cellulose, hydroxyethyl cellulose, agar, gel, proteinaceous polymer, polypeptide, eukaryotic and prokaryotic cells, viruses, lipid, metal, resin, latex, rubber, silicone, e.g., polydimethyldiphenyl siloxane, glass, ceramic, charcoal, kaolinite, bentonite, and the like can also be used. In one embodiment, commercially available Luminex microspheres (Luminex Corp., Austin, Tex.) are used.

Luminex microspheres are extensively discussed in U.S. Pat. No. 6,268,222 and PCT publications WO 99/37814 and WO 01/13120. Briefly, the microspheres are microparticles that incorporate polymeric nanoparticles stained with one or more fluorescent dyes. All of the nanoparticles in a given population are dyed with the same concentration of a dye, and by incorporating a known quantity of these nanoparticles into the microsphere, along with known quantities of other nanoparticles stained with different dyes, a multifluorescent microsphere results. By varying the quantity and ratio of different populations of nanoparticles, it is possible to establish and distinguish a large number of discrete populations of microspheres with unique emission spectra. The fluorescent dyes used are of the general class known as cyanine dyes, with emission wavelengths between 550 nm and 900 nm. These dyes may contain methine groups; the number of methine groups influences the spectral properties of the dye. The monomethine dyes that are pyridines typically have a blue to blue-green fluorescence emission, while quinolines have a green to yellow-green fluorescence emission. The trimethine dye analogs are substantially shifted toward red wavelengths, and the pentamethine dyes are shifted even further, often exhibiting infrared fluorescence emission. However, any dye compatible with the composition of the beads can be used.

When a number of different microbeads or microspheres are used in practicing the methods described herein, it is preferable, but not required, that the dyes have the same or overlapping excitation spectra, but possess distinguishable emission spectra. Multiple classes or populations of particles can be produced from just two dyes. For example, the ratio of nanoparticle populations with red/orange dyes is altered by an adequate increment in proportion so that the obtained ratio does not optically overlap with the former ratio. In this way a large number of differently fluorescing microbead classes are produced.

Detector oligonucleotides may be coupled to the microbead by any suitable means known in the art. The exact method of coupling will vary with the composition of the microbread and the type of linker present, if any. In one embodiment, detector oligonucleotides are coupled to microbeads by the well known carbodiimide coupling procedure. Multiple detector oligonucleotides may be coupled to a single microbead. Microbeads of the same class or group, that is having the same label or fluorescent signature, will have detector oligonucleotides specific for the same target polynucleotide attached to them. The association of the detector oligonucleotide to the target polynucleotide is accomplished using the sensor probes. In some embodiments, more than one group of sensor probe pairs may be directed to a single target polynucleotide. In those cases, the sequence of detector oligonucleotides attached to a single class of microbeads may be different, although all sequences will be associated with the same target polynucleotide.

The detector oligonucleotides are such that at least a portion of the detector oligonucleotide is complementary to a portion of the amplified sensor probe that is, in turn, complementary to the target polynucleotide to allow hybridization of the detector oligonucleotide to the amplified ligated sensor probe. Following denaturation of the amplified sensor probes, if necessary, hybridization is typically performed under moderately stringent, stringent or highly stringent conditions. In one embodiment, hybridization of detector oligonucleotides to amplified sensor probes is performed under stringent conditions. In one embodiment, the portion of the amplified sensor probes that hybridizes with the sensor oligonucleotide comprises the ligation site where the members of the sensor probe pair were ligated prior to amplification. In this manner, conditions can be modified to favor hybridization of detector oligonucleotides to those sensor probe pairs that have been successfully ligated. As is well known in the art, the exact conditions used will vary with the composition of the sensor probes and detector oligonucleotides used. Optimization of hybridization conditions is routine in the art and can be accomplished by the skilled artisan using the guidance provided herein and standard reference texts in molecular biology.

Identification, and if desired quantification, of detector oligonucleotides that have hybridized to ligated and amplified sensor probes is accomplished by means of the detectable labels. Successful hybridization will result in a molecule that comprises both the label associated with the amplified sensor probe and the label associated with the detector molecule. In one embodiment, the presence of each target polynucleotide in the original population of polynucleotides can be determined based on the label associated with the detector oligonucleotide while the amount of each target can be determined by quantifying the amount of sensor probe label associated with each class of detector oligonucleotide. The determination of the labels present and, if desired, their amount can be accomplished using a single measuring device and be accomplished essentially simultaneously, or they can be accomplished using different measuring devices and accomplished in series. For example, detector oligonucleotides can be identified and sorted on the basis of their unique labels. Following sorting, the amount of sensor probe label associated with each class of detector oligonucleotide can be determined, thus allowing the identification and quantification of each target polynucleotide present in the original population of polynucleotides. In another example, a device such as a flow cytometer can be used to individually analyze each detector oligonucleotide and identify it based on its unique marker. The detector oligonucleotide is then analyzed to determined the presence of the amplified sensor probe label. If the sensor probe label is present, an event is recorded for the target polynucleotide associated with that detector molecule.

In one embodiment, detector oligonucleotides and sensor probe labels are differentiated on the basis of differences in emission spectra. In this embodiment, any detection system can be used to detect the difference in spectral characteristics between the two labels, including a solid state detector, photomultiplier tube, photographic film, or eye, any of which may be used in conjunction with additional instrumentation such as a spectrometer, luminometer microscope, plate reader, fluorescent scanner, flow cytometer, or any combination thereof, to complete the detection system.

When differentiation between labels is accomplished by visual inspection, the labels preferably have emission wavelengths of perceptibly different colors to enhance visual discrimination. When it is desirable to differentiate between labels using instrumental methods, a variety of filters and diffraction gratings allow the respective emission maxima to be independently detected.

In one embodiment in which detector oligonucleotides comprise fluorescent microbeads, detector oligonucleotides can be identified using a flow cytometer, for example a fluorescence-activated cell sorter, wherein the different classes of beads in a mixture can be physically separated from each other based on the fluorochrome identity, size and/or shape of each class of bead; and the presence of the target polynucleotide qualitatively or quantitatively determined based on the presence of the detectable label for each sorted pool containing beads of a particular class. Any flow cytometer capable of detecting both the particles and the label contained in the sensor probe hybridized to the detector oligonucleotide can be used. Flow cytometers with multiple excitation lasers and detectors are preferred. In one embodiment the Luminex 100 system is used. As is well known in the art, the exact settings necessary for optimum detection will vary with factors such as the instrument used, the labels used, and the particles used. Optimization of settings and conditions for the use of a flow cytometer for practicing the methods disclosed herein can be accomplished by the skilled technician without undue experimentation. General guidance on the use of flow cytometers can be found in texts such as Shapiro, Practical Flow Cytometry, 3^{rd} ed., Wiley-Liss, 1995 and Jaroszeski et al., Flow Cytometry Protocols, Humana Press, 1998. An example of the use of fluorescent microbeads and flow cytometery can be found in Smith et al., Clin. Chem., 44:2054-2056, 1998. The use of flow cytometry is especially useful in the situation where greater than one class of detector oligonucleotides is used to simultaneously determine the presence of multiple target polynucleotides (multiplex analysis).

The methods described herein can also be used to detect single nucleotide 10. polymorphisms (SNPs). The detection of SNPs can be accomplished with only minor modifications to the methods previously described. For detection of SNPs, a population of polynucleotides is obtained wherein the population contains or is suspected to contain one or more target polynucleotides containing at least one SNP. For detection of SNPs, the pairs of sensor probes are provided wherein a first member of each pair has a 3′ portion that is complementary to the target polynucleotide and the second member has a 5′ portion that is complementary to the target polynucleotide. The sensor probes in each pair are designed so that the 3′ end of the first probe is immediately adjacent to the 5′ end of the second probe when hybridized to the target polynucleotide. In addition, for the detection of SNPs, the terminal nucleotide on either the 3′ end of the first sensor probe or the 5′ end of the second sensor probe is complementary to one of the alleles of the SNP of interest. In one embodiment, different pairs of sensor probes can be used to detect the presence of alternate alleles of the same SNP. In this embodiment, one set of sensor probes have a terminal nucleotide complementary to one allele of the SNP of interest, while the additional sets of sensor probes have terminal nucleotides complementary to the alternate allele or alleles for the SNP of interest.

Following hybridization of the sensor probe pairs to the target polynucleotides under moderately stringent, stringent or highly stringent condtions, the sensor probe pairs are ligated together to form ligated sensor probes under conditions such that if there is a single mismatch at either the 3′ of the first probe or the 5′ end of the second probe, ligation will not occur. The ligated sensor probes are then amplified as described herein such that the amplified sensor probe comprise a detectable label. For each pair of sensor probes used, a detector oligonucleotide having the characteristics previously described is provided. If necessary, the amplified ligated sensor probes are denatured and hybridized to the detector oligonucleotides under moderately stringent, stringent or highly stringent conditions. The hybridization of the detector oligonucleotide to the amplified ligated sensor probes is then determined using any of the methods described herein. The presence, absence or frequency of an allele of the SNP of interest is then determined based on the identity of the dectector oligonucleotide.

The present method allows determination of if a subject is homozygous or heterozygous for a particular allele of the SNP of interest. For example, when sensor probes are provided for only one of two possible alleles, if the allele is not present, there will be no signal above background. If the subject is homozygous for the allele then the maximum signal will be obtained, while if the subject is heterozygous, a signal approximately half that observed with a homozygous individual will be obtained. In another example where sensor probes to each of two alternative alleles are provided, a heterozygous subject will exhibit signals of approximately equal magnitude for both alleles while a homozygous individual will exhibit signals above background for only one of the two alleles.

Because SNPs are increasingly associated with pathological conditions, the methods describe herein can be used for diagnosing a disease, condition, disorder or predisposition associated with a particular SNP or SNPs. In this embodiment, a population of polynucletotides is obtained from a cell or tissue of a test subject. The presence or absence of an allele of at least one SNP of interest in said population of polynucleotides is determined using the methods discussed herein. The presence, absence, or frequency of the SNP allele or alleles of interest in the polynucleotides from the test subject is compared to the presence, absence, or frequency of the allele or alleles or interest in polynucleotides obtained from a reference subject known to have said disease, condition, disorder or predisposition.

The ability to simultaneously determine the presence and/or amount of a multiple target polynucleotides makes the present methods especially suitable for expression profiling. Expression profiling involves the determination of changes in the expression of polynucleotides, e.g. genes, under different conditions and physiological states. Because changes in polynucleotide expression can be related to pathological conditions, the methods described herein are useful for diagnosing a disease, condition, disorder, or predisposition associated with a change in expression patterns. In this aspect, information on the expression of one or more target polynucleotides is obtained from an test subject using the methods described herein and compared to the expression pattern for a subject known to have the disease, condition, disorder, or predisposition of interest. In one embodiment, data representing the expression pattern of a subject with a known disease, condition, disorder or predisposition is stored on a computer readable medium so that the expression pattern from the test subject can be compared to the stored expression pattern.

As used herein, the term “predisposition” refers to the likelihood that an individual subject will develop a particular disease, condition, or disorder. For example, a subject with an increased predisposition will be more likely than average to develop a disease, condition, or disorder, while a subject with a decreased predisposition will be less likely than average to develop a disease, condition, or disorder. The disease, condition, or disorder may be genetic or may be due to a microorganism.

The methods disclosed herein can also be used to determine the developmental or physiological state of a cell or tissue. In this aspect, polynucleotide expression from a test cell or tissue is compared to the expression pattern from a cell of known physiological or developmental state. By comparing the two expression patterns, it is possible to determine the developmental or physiological state of the test cell or tissue. In one embodiment, data representing the expression pattern of a cell or tissue of a known developmental or physiological state is stored on a computer readable medium so that the expression pattern from the test cell or tissue type can be compared to the stored expression pattern.

Yet another aspect provides kits for practicing the novel methods disclosed herein. One embodiment provides a kit comprising, at least one pair of sensor probes for at least one target polynucleotide, at least one detector oligonucleotide for each pair of sensor probes, and instructions for carrying out the novel methods described herein for the determination of polynucleotide expression. Another embodiment provides a kit comprising, at least one pair of sensor probes for at least one target polynucleotide, at least one detector oligonucleotide for each pair of sensor probes, and instructions for carrying out the novel methods described herein for the detection of single nucleotide polymorphisms. In any of the aforementioned kits the detector oligonucleotides can further comprise microbeads or microspheres. Any of the aforementioned kits can also further comprise ligases, primers, dNTPs, polymerase, and/or buffer solutions.

### EXAMPLES

The following examples are intended to provide illustrations of the application of the present invention. The following examples are not intended to completely define or otherwise limit the scope of the invention.

### Example 1

### Plant Material Treatment and RNA Preparation

To illustrate the practice of the method described herein, the expression of the Arabidopsis putative plastocyanin gene was analyzed. This gene is found to be expressed at low levels in the dark, and can be induced by light through the phytochrome A signaling pathway. In the phyA null mutant, the light induction is blocked (Tepperman, et al., Proc. Natl. Acad. Sci. USA, 98:9437-9442, 2001).

Wild-type and phyA null mutant seedlings of Arabidopsis thaliana ecotype RLD were grown on agar plates in the dark condition as described in Tepperman et al., 2001, supra. After 5 days of germination, seedlings were exposed to continuous far-red light (FRc), and whole seedling samples were collected at 7 time points: 0, 1, 3, 6, 12, 18, and 24 hours after the light treatment. An 8^{th} sample was also collected from seedlings maintained in darkness for a further 24 hours (6-day dark controls). RNA samples were prepared as described in Tepperman et al., 2001, supra.

### Example 2

### Design and Synthesis of Oligonucleotides and Probes

All oligonucleotides were designed to minimize secondary structure and dimer formation.

A. Detector Oligonucleotides. Twenty-four-mer detector oligonucleotide sequences were selected for the target mRNA sequences. Since cDNA synthesis is not required in the present method, the detector oligonucleotide sequences can be chosen from any region of mRNA. The Tₘ of the detector oligonucleotides ranged from 65° C. to 75° C. The Tₘ of each half 12 bases ranged from 32° C. to 40° C. The detector oligonucleotides were in the sense direction as the RNA templates, and modified with an amino-Unlinker at 5′ end for coupling to carboxylated microspheres. The oligonucleotides were synthesized by Operon Technologies (Alameda, Calif.), and HPLC purified.

Sensor Probes. Sensor probes were 38 nucleotides in length. Each pair of sensor probes comprised a 20 base sequence on the 3′ end of the first probe and a 20 base sequence on the 5′ end of the second probe that were continuous in sequence and complementary to a continuous 40 nucleotide stretch in the target mRNA. Sensor probes were designed so that after ligation, the center 24 bases would hybridize with the detector oligonucleotides. The final 18 bases on the 5′ end of the first probe and the last 18 bases on the 3′ end of the second probe were common for sensor probes and served as common binding sites for PCR primers. The first probe of each pair was cartridge purified, while the second probe was 5′ phosphorylated and PAGE purified.

C. Primers. The sequence of the primer 1 was the same as the 5′ end portion of the first sensor probe and the sequence of the primer 2 was complementary to the 3′ end of the second sensor primer. The primers were cartridge purified prior to use.

### Example 3

### Conjugation of the Detector Oligonucleotide to Microbeads

Carboxylated fluorochrome microbeads (Luminex, Austin Tex.) were coupled to 5′ amino-Unilinker modified detector oligonucleotides by a one-sep carbodiimide couple procedure following the manufacturer's instructions. The conjugated microspheres were stored in 0.1M MES buffer (2-(N-morpholino) ethane sulfonic acid (Sigma Chemical Co, St. Louis, Mo.), in the dark at a concentration of 5×10⁴ beads/ul.

### Example 4

### Ligation of Sensor Probes

The ligation reaction contained 5-500 ng of total target RNA, and 1 ul of 0.1 uM mixture of each member of a pair of sensor probes in a final volume of 4 ul. For multiplex reactions, all sensor probes were pre-mixed to yield a concentration of 0.1 uM for each sensor probe. One ul of the 0.1 uM pre-mix was then mixed with 500 ng of target RNA to give a final volume of 4 ul. The mixture was heated to 65° C. for 10 minutes, and then slowly cooled to room temperature prior to addition of 2 ul of DNA ligase reaction mixture (1.2 ul 5× buffer, 0.3 ul nuclease-free, and 0.5 ul of T4 DNA ligase, 1 U/ul, Invitrogen, Carlsbad, Calif.). The reaction was incubated at 37° C. for 4 hours, after which the reaction was terminated by heating to 70° C. for 10 minutes and the ligation product was stored at 4° C.

### Example 5

### PCR Amplification of Ligated Sensor Probes

Ligated sensor probes were amplified using two universal primers. Primer 1 had the same sequence as the 5′ common primer binding region located on the first sensor probe of each probe pair and was biotinylated on the 5′ end. The sequence of primer 1 was 5′ Biotin-gctgctagtgtccgatgt 3′ (SEQ ID NO: 1). Primer 2 was complementary to the 3′ common primer binding portion of the second sensor probe of each set and had a sequence of 5′ gatctcctagagtcgtga 3′ (SEQ ID NO: 2). A 20 ul reaction contained 6 ul of ligation product, 1.5 mM MgCl₂, 200 uM of each dNTP, 0.5 uM of each PCR primer, 1 U Taq DNA polymerase (Invitrogen) and lx PCR buffer (Invitrogen). The PCR reaction was carried out in a thermocycler with a hold of 94° C. for 3 minutes, followed by 25 cycles of 94° C. for 30 seconds, 56° C. for 30 seconds and 72° C. for 30 seconds, followed by a hold at 72° C. for 5 minutes.

### Example 6

### Detection

Detector oligonucleotides conjugated to fluorescent microbeads were diluted with 1.5× TMAC buffer (3M tetramethylammonium chloride (TMAC), 0.1% SDS, 50 mM Tris-HCl, pH 8.0, 4 mM EDTA, pH 8.0) at a ratio of 1:330 v/v and preheated to 55° C. In a multiplex reaction, different color-coded (different emission spectra) microbeads, each with a different detector oligonucleotide, were mixed equally in 1.5× TMAC buffer at a ratio of 1:330 (v/v) and pre-heated to 55° C.

Five ul of PCR products were mixed with 12 ul of nuclease-free water and denatured at 95° C. for 10 minutes. Next, 33 ul of 1.5× TMAC buffer diluted microbead conjugated detector olignucleotides were added to the denatured PCR products and mixed. The reaction contained approximately 5000 of each class of detector oligonucleotides. The PCR products were then hybridized with the detector oligonucleotides for 1 hour at 55° C. Following hybridization, the mixture was centrifuged and the supernatant removed. The pellet was resuspended in 80 ul of strepavidin conjugated R-phycoerythrin solution (PE, Molecular Probes, Eugene Oreg.) at 55° C. and incubated in the dark for 10 minutes. Strepavidin conjugated R-phycoerythrin was pre-diluted to 2 ug/ul with 1× TMAC buffer prior to addition to the pellet. Fifty ul of PE stained microbead conjugated detector oligonucleotides from each reaction was analyzed using the Luminex 100 system (Luminex, Austin Tex.) and 100 events counted for each class of detector oligonucleotide. The fluorescence intensity associated with each class of microbead was recorded. The mean fluorescence intensity (MFI) from 100 beads was calculated as a measurement of the relative amount target RNA in the sample.

### Example7

### Specificity

Ligation specificity was examined by studying the effect of different RNA templates, and the effect of different oligonucleotide pairs on their respective mRNA templates in wild-type Arabidopsis seedling total RNA samples. Three phyA-regulated genes were used for this purpose (Table 1A). Expression of these genes should be induced in wild-type Arabidopsis (Tepperman et al., 2001, supra). Four experiments were conducted, the results of which are given in Table 1B.

The first experiment addressed the specificity of the interaction between target RNA and representative sensor probes. The sensor probe pairs were used to detect the expression of Arabidopsis plastocyanin gene in a variety of conditions shown in Table 1A. As predicted, plastocyanin gene transcripts were only detected from light-induced Arabidopsis wild-type seedlings total RNA. When RNA template was omitted, or target RNA was absent in the RNA samples, as in the yeast tRNA sample or mouse lung total RNA sample, the expression signal was similar to the background of water control. A signal, slightly higher than background, was detected from dark-grown Arabidopsis seedling total RNA, indicating a basal level expression of plastocyanin gene in dark-grown seedlings.

The second experiment was designed to examine the specificity of the ligation. Expression of three Arabidopsis genes, a putative plastocyanin, a glycolate oxidase, and a protein with unknown function, was detected using the varied representative sensor probes in a light-grown Arabidopsis seedling total RNA samples. Each gene was represented by sensor probe pairs with perfect match; with one base mismatch at the ligation site; or with mis-paired sensor probes, i.e. one member of the sensor probe pair was homologous not to the target, but different gene. When reactions with perfectly matched sensor probes were used, the expression signals of all three genes studied were detected from the light-grown Arabidopsis seedling total RNA samples. Single-base mismatchs in the sensor probes at the ligation site lowered the detected signal to background levels. Similar results were obtained when mispaired sensor probes were used.

The third experiment examined the importance of the T4 DNA ligase in the ligation reaction. Reactions were conducted with or without T4 DNA ligase. In contrast to the positive control experiment, no signal was detected in the reaction without T4 DNA ligase, clearly demonstrating that the ligase was necessary in the reaction.

The fourth experiment was designed to demonstrate the specificity of hybridization between PCR amplified ligated sensor probes and detector oligonucleotides. In the assay that monitored the expression of three genes, three different microsphere conjugated detector oliognucleotides were mixed prior to hybridization and detection. Only the complimentary sensor probes were picked up by the corresponding detector oligonucleotides, while the hybridization signals of non-matching sensor probes were similar to background

Proper binding of perfectly matched sensor probes to the target mRNA is important for RNA templated T4 DNA ligase mediated sensor probe ligation. The above experiments indicate that T4 DNA ligase can specifically catalyze the ligation of the sensor probes designed to specifically hybridize to the target. The complexity of the non-target RNA in the reaction did not affect the specificity of the ligation, as demonstrated by the results from Arabidopsis and mouse lung samples. The ligation reaction did not tolerant the non-specific sensor probes, such as single-base mismatched sensor probe pairs and mis-paired sensor probes, even if they are adjacently located by non-specific binding to the target RNA. The ligation also required specific target polynucleotides to be presented in the reaction. Without RNA or without target mRNA, the oligonucleotide pair was not ligated by T4 DNA ligase.

### Example 8

### Sensitivity and Linear Range

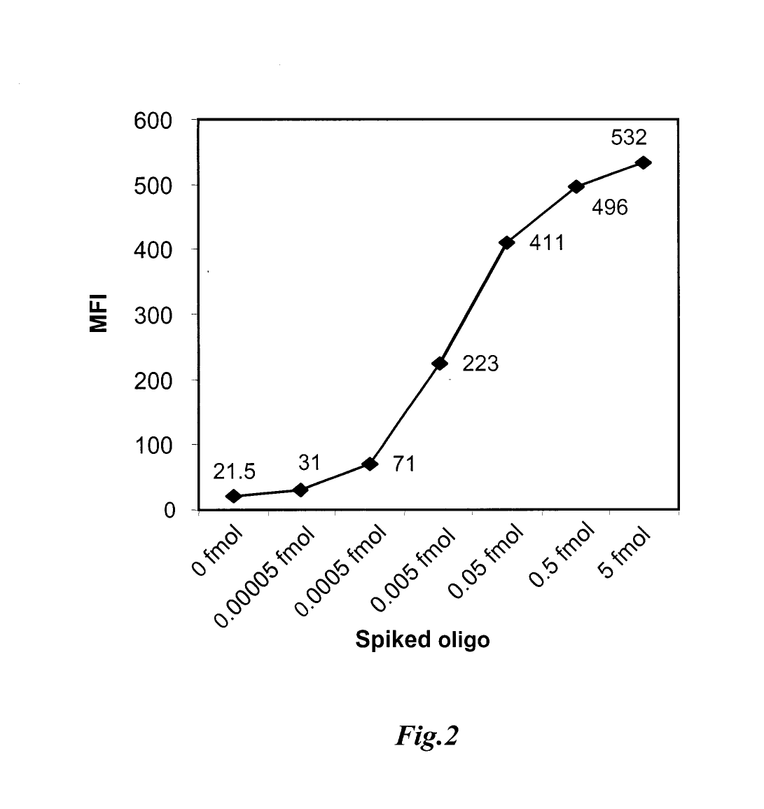

A spike-in experiment was conducted to determine the sensitivity of the present methods. In a background of 500 ng of mouse lung total RNA, a 40-base oligonucleotide putative plastocyanin gene sequence was added at a serial dilution of 5, 0.5, 0.05, 0.005, 0.0005, 0.00005 and 0 fmole. The sensor probe pair (probes 1 and 2), PCR primers and detector oligonucleotides were identical to the previous plastocyanin experiment and are listed in Table 1A. While the signal detected from mouse lung total RNA without addition of template oligonucleotide was similar to background, signals could be clearly detected for amounts as low as 0.00005 fmole of spiked oligonucleotides in a background of 500 ng mouse lung total RNA (FIG. 2). In a 5 ul reaction volume, 0.00005 fmole of targets has an approximate concentration of 1×10⁻⁴M. If the average length of a RNA molecule is presumed to be 1000 bases and the molecular weight of a nucleotide is 330, a 5 ul reaction volume of 500 ng total RNA is equivalent to 3×10⁻⁷ M. Thus, the method detected a specific target from a complex RNA population with the molecular ratio of 1:3×10⁷. If each cell contains 10⁵ mRNA molecules and there are total 10⁷ RNA molecules per cell (assuming that mRNA is 1% of total RNA and the molecular weight of different RNA species is same), 1 of 3×10⁷ equates to less than one copy of mRNA target/cell. If calculated by weight, 0.00005 fmole is equivalent to 16.5 pg for a 1 kb long mRNA molecule. Thus the method can detect as low as 16.5 pg mRNA target in a 500 ng total RNA sample. If a target RNA has more copies in a cell, the required total RNA amount will be reduced. For example, the previous data showed the expression level of putative plastocyanin gene in dark-grown wild type Arabidopsis seedling is just above the background, the present method detected its expression in 500 ng of total RNA, while in light-grown Arabidopsis, 5 ng of RNA was enough for detection.

### Example 9

### Dynamic Range

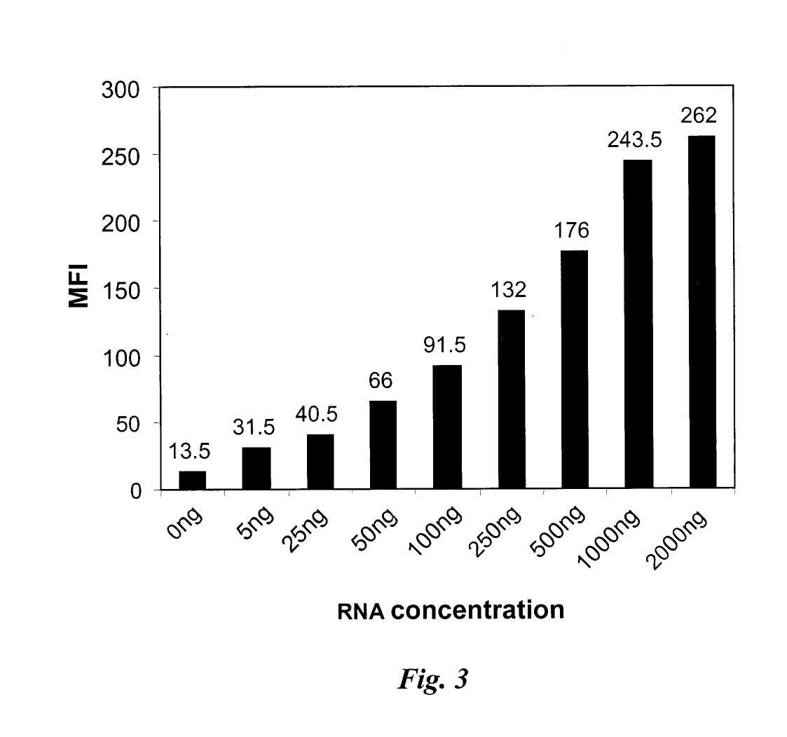

Dynamic range is contributed by both PCR amplification of the DNA oligonucleotide sensors and dynamic range of the label associated with amplified ligated sensor probes. In order to achieve the linear amplification, PCR reactions are generally limited to 25-30 cycles. So the detection dynamic range observed is mainly determined by the dynamic range of the sensor probe readout. Based on the above sensitivity study, the signal intensity of the method disclosed herein has a linear response to the spike-in template amount between the range of 0.0005 fmole and 0.05 fmole. The dynamic range in response to different RNA concentrations was determined using total RNA from Arabidopsis light-grown wild-type seedlings in a serial concentration from 5 ng-2 ug as templates. The putative plastocyanin gene transcript was selected as the target. With a 30-cycle PCR reaction, the plastocyanin gene transcript could be detected from as low as 5 ng of total RNA and the signal intensity qualitatively increased as the amount of starting total RNA increased (FIG. 3). The linear increase of signal intensity was observed within 5 ng-1 ug of total RNA. The signal intensity change was correlated with the absolute RNA concentration, while the change ratio of signal intensity declined when the total RNA amount was over 1 ug. The saturation of signal intensity was mainly limited by the detection dynamic range of liquid array. No signal was detected from as much as 500 ng of the control mouse lung total RNA sample. An increase in PCR cycles beyond 35 did not significantly increase the signal, as the PCR amplification often reached the plateau at 35 cycles (data not shown). The increase in signal intensity was from the increase in the amount of PCR amplified DNA sensor probes.

### Example 10

### Reproducibility

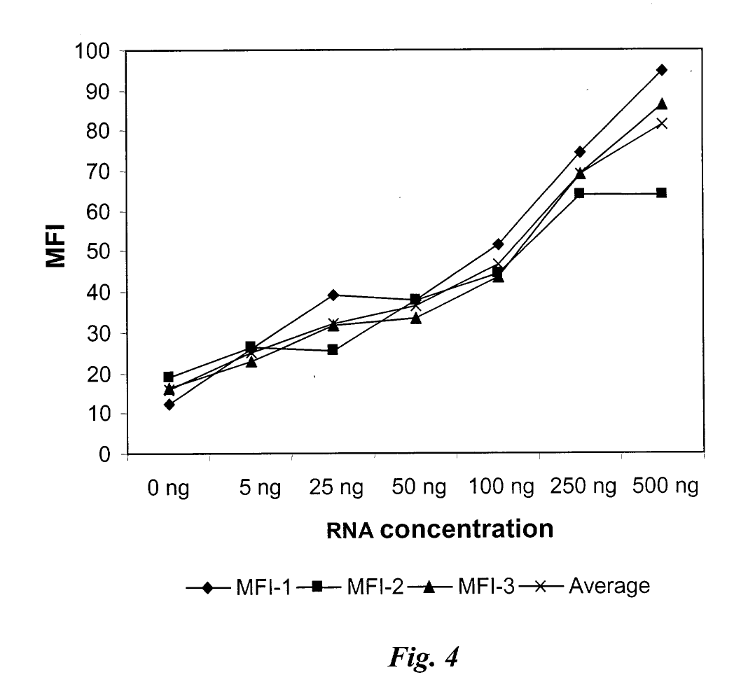

The reproducibility was characterized by conducting 3 replicate experiments using the same RNA sample in serial dilutions. In this experiment, sensor production by ligation reaction, sensor amplification by PCR, and sensor hybridization and detection were conducted independently with 5-500 ng total RNA in each assay. On average, less than a 14% of coefficient of variance (CV) was observed among all samples. The CVs among three replicates in different dilution concentrations ranged from 2% to 21% (FIG. 4), and accounted for the variation of sensor preparation and the variation of hybridization beads preparation. In practice, all reactions in one experiment will share the same master mix of reagents and microspheres, thus the overall variation among samples will be reduced compared to this reproducibility assay. Since the variation among replicates are generally less than 20%, the signal intensity with greater than 2-fold differences between samples can be safely interpreted as an expression difference.

### Experiment 11

### Multiplex Detection

To demonstrate multiplex detection, the expression of 11 phyA-regulated Arabidopsis genes and the control housekeeping gene polyubiquitin were monitored simultaneously. Sixteen RNA samples of wild-type and phyA-null mutant used in the previous study to profile the expression changes in a time course of light treatments were used in this study for direct comparison. A total of 12 pairs of sensor probes were mixed with each total RNA sample to represent the 12 target mRNA transcripts. Correspondingly, 12 kinds of color-coded microspheres with respective detector oligonucleotides were mixed in the hybridization for simultaneous detection.

As predicted, the polyubiquitin gene showed a consistent expression over the time course in both wild-type and mutant, suggesting that it is neither phyA-regulated, nor light sensitive. Five photosynthesis related genes, including putative plastocyanin, magnesium chelatase subunit, chloroplast FtsH protease, and 2 representative sequences of photosystem II type I chlorophyll a/b binding protein, were quickly induced in the wild-type seedlings in the first two hours after transfer to FRc, and maintained at the high expression level over 24 hour time period, but were completely repressed in phyA null mutant. The transcription level of PhyA suppressor spal increased quickly in the early stage, and decreased its expression to the basal level later. Two genes related to cellular metabolism and stress and defense response (glycolate oxidase and a gene similar to SOR 1 from fungus Cercospora nicotianae), and an unknown protein were induced at a later stage in the wild-type (three hours after exposing to light). Two of the genes surveyed were repressed by the light treatment in the wild-type, but the depression could be released by phyA-null mutation. One of them encodes a protein highly similar to branched-chain amino acid amino transferase, and the other encodes a protein responsible for fatty acid elongation from C28 to C30.

Compared to the previous results from GeneChip microarrays, 11 of 12 genes studied showed a very high correlation with almost identical expression patterns. The rest had a similar expression pattern over the time course; however, the amplitude of the change was less. The average correlation coefficient between the two sets of data was 0.86 with a standard deviation of 0.18. Based on these results, it can be seen that results using the novel methods described herein and GeneChip microarrays are consistent, despite of the completely different strategies and principles between the two methods. These results also show that the present methods are valid for quantification of differential gene expression since the variation detected could be confirmed by other well-established methods.

TABLE I

| | | | | |
|---|---|---|---|---|
| Gene | | | | |
| ID | Gene Name | 3′ End 1^{st} Sensor Probe | 5′ End 2^{nd} Sensor Probe | Detector Oligo |

| | | | | |
|---|---|---|---|---|
| 12078 | glycolate | gcttgaatcgctatccttgc(a) | patcctctcctgtaagaacac | [Am-Uni]-acaggagaggatgcaaggatagcg |
| | oxidase | SEQ ID NO:3 | SEQ ID NO:4 | SEQ ID NO:5 |
| 12739 | unknown | agcccaccaccagactcttc(g) | patcgtctttcttagccgccg | [Am-Uni]-aagaaagacgatgaagagtctggt |
| | protein | SEQ ID NO:6 | SEQ ID NO:7 | SEQ ID NO:8 |
| 13234 | putative | gagatgacttcactgtaagc(g) | pttcggtgatgcaacagcagc | [Am-Uni]-tgcatcaccgaagcttacagtgaa |
| | plastocyanin | SEQ ID NO:9 | SEQ ID NO:10 | SEQ ID NO:11 |

* In mis paired oligos, oligos I of genes 12078, 12739 and 13234 were paired with oligos II of genes 13234, 12078 and 12739, respectively.
* In mis mached oligo pairs, the last base at 3′ end of oligo I was substituted by the base in the parenthesis.

TABLE 1B
Mean Fluorescence Intensity from Specific Treatments

| | | MFI | | |
|---|---|---|---|---|
| GENE | TREATMENT | 12078 | 12781 | 13234 |
| | H₂O instead of PCR products in detection step | | | 13 |
| | No RNA | | | 12 |
| | Yeast tRNA | | | 23 |
| | Mouse lung total RNA | | | 12 |
| | Arabidopsis total RNA (grown in dark) | | | 33 |
| | Arabidopsis total RNA (induced in light) | | | 159 |
| 12078 | Perfectly matched sensor probe pair | 220 | 17 | 16 |
| | Perfectly matched sensor probe pair w/o ligase | 15 | 32 | 7 |
| | Mismatched sensor probe pair | 21 | 10 | 23 |
| | Mis-paired sensor probe pair | 15 | 11 | 13 |
| 12781 | Perfectly matched sensor probe pair | 15 | 94 | 13 |
| | Perfectly matched sensor probe pair w/o ligase | 13 | 14 | 14 |
| | Mismatched sensor probe pair | 15 | 21 | 13 |
| | Mis-paired sensor probe pair | 14 | 15 | 12 |
| 13234 | Perfectly matched sensor probe pair | 12 | 12 | 232 |
| | Perfectly matched sensor probe pair w/o ligase | 20 | 13 | 5 |
| | Mismatched sensor probe pair | 11 | 13 | 20 |
| | Mis-paired sensor probe pair | 16 | 10 | 10 |

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

it is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for determining polynucleotide expression comprising:
providing a population of polynucleotides, wherein said population comprises at least one target polynucleotide comprising a known nucleotide sequence;
providing for each target polynucleotide, a pair of specific sensor probes, each probe in said pair having a 3′ end and a 5′ end, a first probe of said pair having a 3′ portion that is complementary to said target polynucleotide and a 5′ portion comprising a common primer binding site, and a second probe of said pair having a 5′ portion complementary to said target polynucleotide and a 3′ portion comprising a common primer binding site, wherein said complementary portions on said sensor probes are immediately adjacent on said target polynucleotide;
combining said at least one polynucleotide target with its pair of specific sensor probes under stringent hybridization conditions;
allowing said sensor probes to hybridize to said at least one target polynucleotide;
ligating hybridized members of a pair of sensor probes to form a ligated sensor probe;
amplifying said ligated sensor probes to provide amplified ligated sensor probes wherein said amplified ligated sensor probes comprise a detectable label;
for each different pair of sensor probes providing at least one class of detector oligonucleotide, said detector oligonucleotide comprising a detectable label that is different for each class of detector oligonucleotide and capable of being differentiated from the label of said amplified ligated sensor probes, wherein said detector oligonucleotide is capable of hybridizing to a portion of said ligated sensor probes that is complementary to said target polynucleotide;
combining said labeled amplified ligated sensor probes with said detector oligonucleotides under stringent conditions and allowing said detector oligonucleotides to hybridize to said amplified ligated sensor probes;
determining the hybridization of said detector oligonucleotides to said amplified ligated sensor probes by detecting the presence of said detectable label of said detector oligonucleotide in association with said detectable label of said amplified ligated sensor probes; and
identifying said target polynucleotide by the identity of the detector oligonucleotide.

2. The method of claim 1, wherein said detector oliognucleotide further comprises a microsphere, said microsphere comprising said detectable label for said detector oligonucleotide.

3. The method of claim 2, wherein said microbead is a Luminex microsphere.

4. The method of claim 1, wherein said detectable labels are detected by a flow cytometer.

5. The method of claim 1, wherein said determination of said detectable labels is quantitative.

6. The method of claim 1, wherein said ligation is achieved using a T4 ligase.

7. The method of claim 1, wherein said at least one target polynucleotide is obtained from a plant or an animal.

8. The method of claim 1, wherein said at least one target polynucleotide comprises DNA

9. The method of claim 1, wherein said at least one target polynucleotide comprises RNA.

10. The method of claim 1, wherein said detector oligonucleotides hybridizes to a portion of said ligated sensor probes that comprises said 3′ end of said first sensor probe and said 5′ end of said second sensor probe.

11. The method of claim 1, wherein said amplification uses primers that bind to said common primer binding sites.

12. The method of claim 11, wherein said amplification is by the polymerase chain reaction.

13. The method of claim 1, wherein said population of polynucleotides comprises at least 20 different target polynucleotides.

14. The method of claim 1, wherein said population of polynucleotides comprises at least 50 different target polynucleotides.

15. The method of claim 1, wherein said population of polynucleotides comprises at least 100 different target polynucleotides.

16. The method of claim 1, wherein said detector oligonucleotides are from about 18 nucleotides long to about 30 nucletotides long.

17. The method of claim 1, wherein said portion of each sensor probe in a sensor probe pair that is complementary to the target polynucleotide is from about 15 nucleotides long to about 30 nucleotides long.

18. The method of claim 17, wherein said portion of each sensor probe in a sensor probe pair that is complementary to the target polynucleotide is about 20 nucleotides long.

19. The method of claim 1, wherein said common primer binding site is from about 12 nucleotides long to about 24 nucleotides long.

20. The method of claim 19, wherein said common primer site is about 18 nucleotides long.

21. The method of claim 1, wherein each of said sensor probes comprise a first portion complementary to said target polynucleotide and said detector oligonucleotide; a second portion complementary to said target polynucleotide, but not complementary to said detector oligonucleotide; and a third portion comprising a common primer binding site that is not complementary to either said target polynucleotide or said detector polynucleotide.

22. The method of claim 21, wherein when hybridized to said target polynucleotide, said first portions of each of the sensor probes in a sensor probe pair are adjacent to each other.

23. A method for detecting a single nucleotide polymorphism comprising:
providing a population of polynucleotides, wherein said population comprises at least one target polynucletotide containing a single nucleotide polymorphism;
providing for each target polynucleotide at least one pair of specific sensor probes, each probe in said pair having a 3′ end and a 5′ end, a first sensor probe of said pair having a 3′ portion that is complementary to said target polynucleotide and a 5′ portion comprising a common primer binding site, and a second sensor probe of each pair having a 5′ portion that is complementary to said target polynucleotide and a 3′ portion comprising a common primer binding site, wherein said complementary portions on said sensor probes in said pair are immediately adjacent on said target polynucleotide and either the 3′ end of said first probe or the 5′ end of said second probe is complementary to an allele of said single nucleotide polymorphism;
combining said at least one polynucleotide target with its at least one pair of specific sensor probes under stringent hybridization conditions;
allowing said sensor probes to hybridize to said at least one target polynucleotide;
ligating hybridized members of a pair of sensor probes to form a ligated sensor probe under conditions such that if the single nucleotide polymorphism allele present is not complementary to said sensor probes then ligation does not occur;
amplifying said ligated sensor probes to provide amplified ligated sensor probes wherein said amplified ligated sensor probes comprise a detectable label;
for each pair of different sensor probes providing at least one class of detector oligonucleotide, said oliognucleotides comprising a detectable label that is different for each class of detector oligonucleotide and capable of being differentiated from the label of said amplified ligated sensor probes, wherein said detector oligonucleotides are capable of hybridizing to a portion of said ligated sensor probe that is complementary to said target polynucleotide;
combining said amplified ligated sensor probes with said detector oligonucleotides under stringent conditions and allowing said detector oligonucleotides to hybridize to said amplified ligated sensor probes;
determining the hybridization of said detector oligonucleotides to said amplified ligated sensor probes by detecting the presence of said detectable label of said detector oligonucleotides in association with said detectable label of said amplified ligated sensor probes; and
determining the presence, absence or frequency of said allele of said single nucleotide polymorphism, said allele identified by the detector oligonucleotide label.

24. The method of claim 23 further comprising, providing for each target polynucleotide at least a first and a second pair of specific sensor probes wherein either the 3′ end of said first probe or the 5′ end of said second probe of said first pair of sensor probes is complementary to an allele of said single nucleotide polymorphism, and either the 3′ end of said first probe or the 5′ end of said second probe of said second pair of sensor probes is complementary to a different allele of said single nucleotide polymorphism.

25. The method of claim 23, wherein said detector oliognucleotide further comprises a microsphere, said microsphere comprising said detectable label for said detector oligonucleotide.

26. The method of claim 25, wherein said microsphere is a Luminex microsphere.

27. The method of claim 23, wherein said labels are detected by flow cytometry.

28. The method of claim 23, wherein said determination of the hybridization of said detector oligonucleotides to said amplified ligated sensor probes is quantitative.

29. The method of claim 23, wherein said ligation is achieved using a T4 ligase.

30. The method of claim 23, wherein said at least one target polynucleotide is obtained from a plant or an animal.

31. The method of claim 23, wherein said at least one target polynucleotide comprises DNA

32. The method of claim 23, wherein said at least one target polynucleotide comprises RNA.

33. The method of claim 23, wherein said detector oligonucleotide hybridizes to a portion of said ligated sensor probes that comprises said single nucleotide polymorphism.

34. The method of claim 23, wherein said amplification uses primers that bind to said common primer binding sites.

35. The method of claim 34, wherein said amplification is by the polymerase chain reaction.

36. The method of claim 23, wherein said population of polynucleotides comprises at least 20 different target polynucleotides.

37. The method of claim 23, wherein said population of polynucleotides comprises at least 50 different target polynucleotides.

38. The method of claim 23, wherein said population of polynucleotides comprises at least 100 different target polynucleotides.

39. The method of claim 23, wherein said portion of each sensor probe in a sensor probe pair that is complementary to the target polynucleotide is from about 15 nucleotides long to about 30 nucleotides long.

40. The method of claim 39, wherein said portion of each sensor probe in a sensor probe pair that is complementary to the target polynucleotide is about 20 nucleotides long.

41. The method of claim 23, wherein said common primer binding site is from about 12 nucleotides long to about 24 nucleotides long.

42. The method of claim 41, wherein said common primer site is about 18 nucleotides long.

43. The method of claim 23, wherein said detector oligonucleotide is from about 18 nucleotides long to about 30 nucleotides long.

44. The method of claim 23, wherein each of said sensor probes comprises a first portion complementary to said target polynucleotide and said detector oligonucleotide; a second portion complementary to said target polynucleotide, but not complementary to said detector oligonucleotide; and a third portion comprising a common primer binding site that is not complementary to either said target polynucleotide or said detector oligonucleotide.

45. The method of claim 44, wherein when hybridized to said target polynucleotide, said first portions of each of the sensor probes in a sensor probe pair are adjacent to each other.

46. A method for determining the physiological or developmental state of a cell or tissue comprising,
obtaining a population of polynucleotides from a test cell or test tissue of an unknown physiological or developmental state, wherein said population comprises at least one target polynucleotide of interest;
determining expression of said at least one target polynucleotide of interest by the method of claim 1; and
comparing expression of said at least one target polynucleotide from said test cell or tissue to expression of said at least one target polynucleotide obtained from a reference cell or tissue of a known physiological or developmental state also determined by the method of claim 1.

47. A method of diagnosing a disease, condition, disorder, or predisposition comprising:
obtaining a population of polynucletotides from a cell or tissue of a test subject, wherein said population comprises at least one target polynucleotide of interest;
determining expression of said at least one target polynucleotide of interest by the method of claim 1; and
comparing expression of said at least one target polynucleotide of interest from said subject to the expression of said at least one target polynucleotide of interest obtained from a reference subject known to have said disease, condition, disorder, or predisposition also determined by the method of claim 1.

48. A method of diagnosing a disease, condition, disorder, or predisposition associated with a single nucleotide polymorphism (SNP) comprising:
obtaining a population of polynucletotides from a cell or tissue of a test subject;
determining the presence, absence or frequency of an allele of at least one SNP of interest in said population of polynucleotides using the method of claim 23; and
comparing the presence, absence or frequency of said allele in the population of polynucleotides from said test subject to the presence, absence or frequency of said allele in a population of polynucleotides obtained from a reference subject known to have said disease, condition, disorder, or predisposition.

49. A method of diagnosing a disease, condition, disorder, or predisposition associated with a single nucleotide polymorphism (SNP) comprising:
obtaining a population of polynucletotides from a cell or tissue of a test subject;
determining the presence, absence or frequency of at least one allele of at least one SNP of interest in said population of polynucleotides using the method of claim 24; and
comparing the presence, absence or frequency of said allele in the population of polynucleotides from said test subject to the presence, absence or frequency of said allele in a population of polynucleotides obtained from a reference subject known to have said disease, condition, disorder, or predisposition.

50. A kit, comprising at least one pair of sensor probes for at least one target polynucleotide, at least one detector oligonucleotide for each pair of sensor probes, and instructions for carrying out the method of claim 1.

51. The kit of claim 50, wherein said at least one detector oligonucleotide further comprises a labeled microbead or microsphere.

52. A kit, comprising at least one pair of sensor probes for at least one target polynucleotide, at least one detector oligonucleotide for each pair of sensor probes, and instructions for carrying out the method of claim 23.

53. The kit of claim 52, wherein said at least one detector oligonucleotide further comprises a labeled microbead or microsphere.

54. A kit, comprising at least one pair of sensor probes for at least one target polynucleotide, at least one detector oligonucleotide for each pair of sensor probes, and instructions for carrying out the method of claim 24.

55. The kit of claim 54, wherein said at least one detector oligonucleotide further comprises a labeled microbead or microsphere.
